(19) United States
(12) Patent Application Publication
(10) Pub. No.: US 2013/0138016 A1
(43) Pub. Date: May 30, 2013

(54) METHOD OF GENERATING AN ALERT WHILE IMAGING A BREAST OF A SUBJECT DURING A MAMMOGRAM

(71) Applicant: General Electric Company, Schenectady, NY (US)

(72) Inventors: Raja Shekhara, Karnataka (IN); Raghavan Ranganathan, Bangalore (IN)

(73) Assignee: General Electric Company, Schenectady, NY (US)

(21) Appl. No.: 13/678,592

(22) Filed: Nov. 16, 2012

(30) Foreign Application Priority Data
Nov. 30, 2011 (IN) 4161/CHE/2011

(51) Int. Cl. A61B 5/00

(52) U.S. Cl. CPC A61B 5/00
USPC 600/587

(57) ABSTRACT

A method of generating an alert while imaging a breast of a subject is provided. The method comprising applying a predefined pressure on the breast holder, confining the breast in a predefined field of view, and generating an alert based on at least one predefined condition.

### BACKGROUND OF THE INVENTION

The subject matter disclosed herein relates to providing alerts during mammography. More particularly, the disclosure relates to a method and system to alert a user to adjust the position of a person's breast during a mammogram.

Mammography has earned a great deal of significance, as it is a method for detecting signs of breast cancer. To perform a mammography, a patient's breast needs to be aligned properly with the X-ray source and the image receptor. It involves positioning a patient's breast in a desired orientation against a horizontal wall and compressing the breast. The breast is then exposed to X-rays to create a latent image of the breast on the image receptor. In today's scenario, whenever a Mammography patient undergoes X-ray examination, technicians performing the operation in the clinical setting need to spend a significant amount of time in adjusting the position of the patient's breast in order to achieve the desired orientation.

In cases where patients are suffering with diseases like obesity and kyphosis, the patients have a tendency to move away from chest wall during compression. Also, due to the time taken for compression, the patient may move away slightly. This will result in incomplete posterior breast tissue coverage.

Further, during compression, the breast may move outside the field of view in which X-rays are passed. Due to this, some tissue will not be included, and therefore, the complete breast will not be within the detector limits, which in turn will require additional views. Further, it will require recompression and repositioning of the patient. This also results in patients being exposed to multiple shots of X-ray radiation which is undesirable and is considered harmful.

It would be desirable to have a mammography system to overcome the above-mentioned problems.

### BRIEF DESCRIPTION OF THE INVENTION

The shortcomings of the prior art are overcome and additional advantages are provided through the provision of a method and a system as described in the description.

The present disclosure relates to a system and method for positioning a breast of a subject during mammogram.

According to an embodiment of the disclosure, a method of generating an alert while imaging a breast of a subject is provided. The method comprising applying a predefined pressure on the breast holder, confining the breast in a predefined field of view, and generating an alert based on at least one predefined condition.

According to another embodiment of the disclosure, a mammographic system is provided. The mammographic system comprises at least one sensor configured to determine a current pressure applied on a breast holder while imaging, a processor configured to compare the current pressure and a predefined pressure, and an indicator configured to alert an operator of the system based on the comparison of the current pressure and the predefined pressure.

According to yet another embodiment of the invention, a mammographic system is provided. The system comprises at least one sensor configured to identify a current field of view of a compressed breast while imaging, a processor configured to compare the current field of view and a predefined field of view, and an indicator configured to alert an operator of the system based on the comparison of the current field of view and the predefined field of view.

The aforementioned and other features and advantages of the invention will become further apparent from the following detailed description of the presently preferred embodiments, read in conjunction with the accompanying drawings. The detailed description and drawings are merely illustrative of the disclosure rather than limiting, the scope of the disclosure being defined by the appended claims and equivalents thereof.

### BRIEF DESCRIPTION OF THE DRAWINGS

The features of the present disclosure are set forth with particularity in the appended claims. The disclosure itself, together with further features and attended advantages, will become apparent from consideration of the following detailed description, taken in conjunction with the accompanying drawings. One or more embodiments of the present disclosure are now described, by way of example only, with reference to the accompanied drawings wherein like reference numerals represent like elements and in which:

FIG. 1 illustrates a block diagram of a mammography system in accordance with an embodiment of the present disclosure;

FIG. 2 illustrates a device for sensing pressure during mammography in accordance with an embodiment of the present disclosure;

FIG. 3 illustrates a device with a position sensing mechanism in accordance with an embodiment of the present disclosure; and

FIG. 4 illustrates a block diagram of photoelectric sensors in accordance with an embodiment of the present disclosure.

### DETAILED DESCRIPTION OF THE INVENTION

The figures depict embodiments of the disclosure for purposes of illustration only. One skilled in the art will readily recognize from the following description that alternative embodiments of the structures and methods illustrated herein may be employed without departing from the principles of the disclosure described herein.

The foregoing has broadly outlined the features and technical advantages of the present disclosure in order that the detailed description of the disclosure that follows may be better understood. Additional features and advantages of the disclosure will be described hereinafter, which form the subject of the claims of the disclosure. It should be appreciated by those skilled in the art that the conception and specific aspect disclosed may be readily utilized as a basis for modifying or designing other structures for carrying out the same purposes of the present disclosure. It should also be realized by those skilled in the art that such equivalent constructions do not depart from the spirit and scope of the disclosure as set forth in the appended claims. The novel features which are believed to be characteristic of the disclosure, both as to its organization and method of operation, together with further objects and advantages, will be better understood from the following description when considered in connection with the accompanying figures. It is to be expressly understood, however, that each of the figures is provided for the purpose of illustration and description only and is not intended as a definition of the limits of the present disclosure.

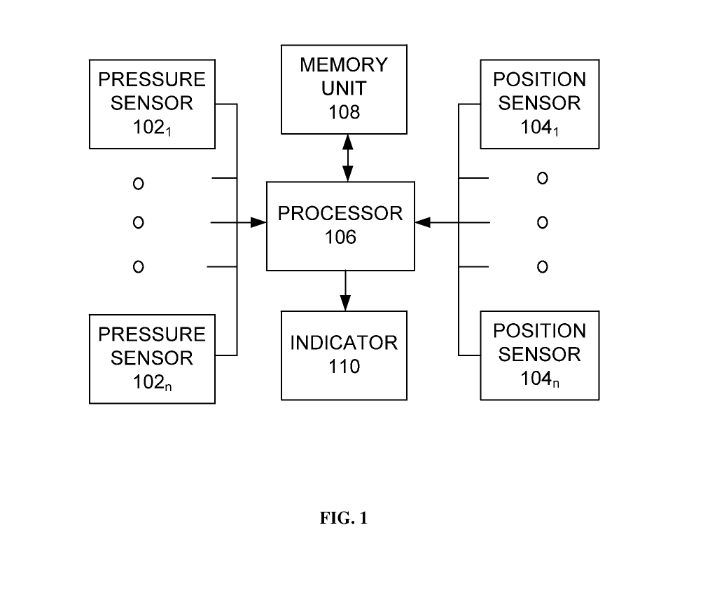

FIG. 1 illustrates a block diagram of various components of a mammography system in accordance with an embodiment of the present disclosure.

The mammography system is used for imaging a breast of a patient (alternatively referred to as subject) to scan for cancer cells. For this, complete coverage of the breast would be required so that each tissue of the breast is scanned. The present disclosure relates to providing an alert to the operator of the system if the breast is not fully covered. For this, the system comprises at least one pressure sensor (102) and at least one position sensor (104) connected to a processor (106). In an exemplary embodiment, FIG. 1 shows a plurality of pressure sensors (102₁. . . 102ₙ), and a plurality of position sensors (104₁. . . 104ₙ). The system further comprises a memory unit (108) and an indicator (110). In an exemplary embodiment, the memory unit stores predefined values of pressure, predefined field of views, angles defining predefined field of views and physical dimensions of patients. The sensor (102) determines a current pressure applied on the breast holder while imaging. The processor (106) then compares this current pressure with predefined pressure stored in the memory unit (108). If there is a mismatch, the operator is alerted by the indicator (110) which displays an LED of red color. This alert requires the operator to apply appropriate force on the patient. Otherwise, if the compared values match, a green color LED is displayed on the indicator (110) which indicates that the operator can now take an image of the patient. However, light sources of any color can be used for indication. In an embodiment an audio signal could be used for indication. In another embodiment, both visual and audio signals could be used as alert indicators.

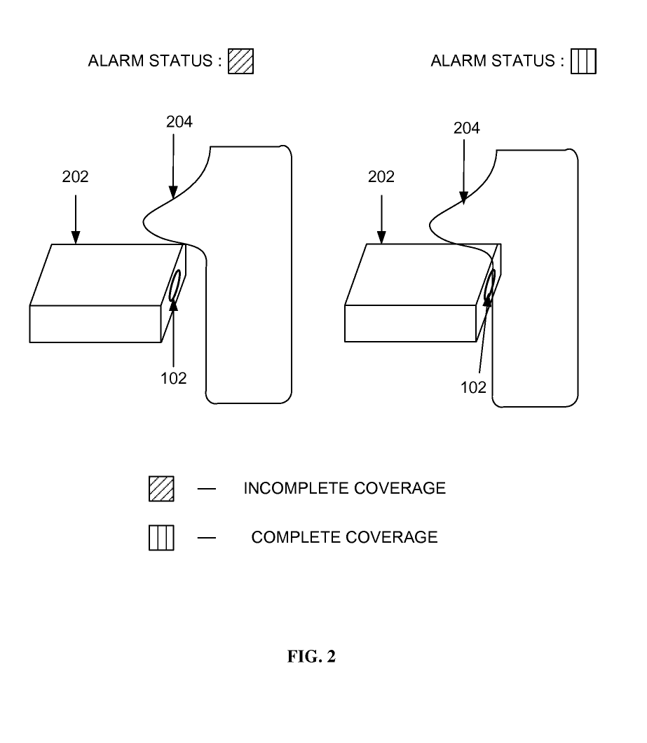

FIG. 2 illustrates a device for sensing pressure during mammography in accordance with an embodiment of the present disclosure.

When a patient is subjected to imaging under mammographic procedure, the operator has to apply some pressure onto the patient in order to bring the breast under field of view completely.

The amount of pressure to be applied varies for each patient depending on data including, but not limited to height, weight and breast size.

In an embodiment, the pressure value is computed either automatically or manually from the physical dimensions of patient. The dimensions of the patient can be either obtained by measuring physical dimensions of the patient or using dimensions already stored in memory. In an embodiment, generic patient information can also be used to compute the pressure to be applied on a patient. The physical dimensions of the patient can be stored in at least one of an internal and an external memory.

FIG. 2 shows a breast holder (202) of a mammography system. In an embodiment, the breast holder can be a chest wall. The chest wall (202) further comprises a pressure sensor (102) disposed on an edge of the chest wall (202). Breast (204) of the patient is pushed towards the breast holder (202). The pressure sensor (102) constantly measures the pressure applied on the edge of the chest wall by the patient. Then the current measured pressure value is compared with the predefined pressure value already stored. Upon comparison, an alert is generated as an indication. In an embodiment, visual indication is used to alert an operator of the mammography system. If the current measured pressure value matches the pressure value, a green color LED is lit on the indicator of the mammography system. This indicates that the operator of the system can now scan the breast of the patient. And, if the current measured pressure value does not match the threshold pressure value, a different color LED other than green is lit on the indicator of the mammography system. This indicates that the breast of the patient should be pushed further to put more pressure on the chest wall (202). A person skilled in the art would understand that LEDs of any color can be used to alert the operator of the system.

In another exemplary embodiment, an audio alert can be provided to the operator. Even a combination of audio and visual indication can be provided to alert the operator.

A person skilled in the art would understand that any number of pressure sensors (102) can be used in the pressure sensing mechanism according to the requirement.

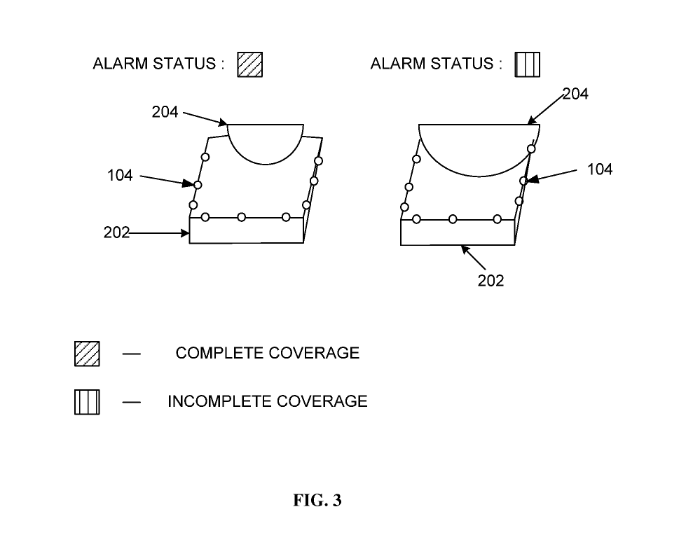

FIG. 3 illustrates a device with a position sensing mechanism in accordance with an embodiment of the present disclosure.

When a patient is subjected to imaging under mammographic procedure, the breast (204) of the patient should be properly placed on the chest wall (202). The breast (202) of the patient should be placed within a field of view and compressed in order to examine each tissue of the breast clearly. The predefined field of view is predefined based on standard field of views as exists today in mammography X-ray system. The predefined field of view for each patient depends on the physical dimensions of the patient.

Position sensors (104) are placed on the surface of the chest wall (202). In an exemplary embodiment, the position sensor is a photoelectric sensor. The sensor (104) senses the position of the breast (204) placed on the chest wall (202) to determine a current field of view. A person skilled in the art would understand that apart from photoelectric sensors, any other similar sensors can be used in the present disclosure. Then, the current field of view of the compressed breast is compared with the predefined field of view already stored. Upon comparison, an alert is generated as an indication. In an embodiment, visual indication is used to alert an operator of the mammography system. If the current field of view matches the predefined field of view, a green color LED is lit on the indicator of the mammography system. This indicates that the operator of the system can now scan the breast of the patient. In another embodiment, if the current field of view does not match the predefined field of view, a different color LED other than green is lit on the indicator of the mammography system. This indicates that the breast (204) of the patient should be adjusted and placed within the predefined field of view. A person skilled in the art would understand that LEDs of any color can be used to alert the operator of the system.

In another exemplary embodiment, an audio alert can be provided to the operator upon comparison of the current field of view with the predefined field of view. Even a combination of audio and visual indication can be provided to alert the operator.

A person skilled in the art would understand that any number of position sensors (104) can be used in the pressure sensing mechanism according to the requirement.

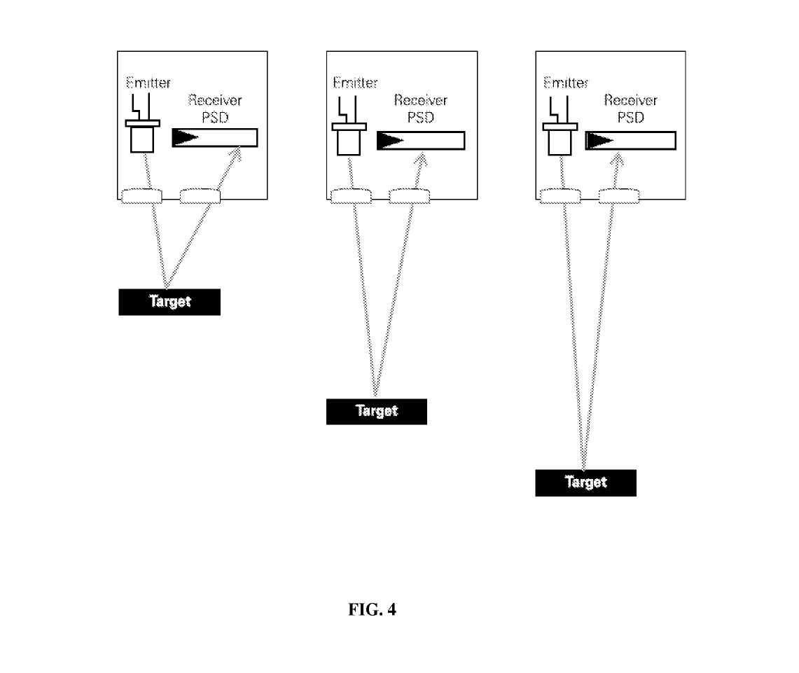

FIG. 4 illustrates a block diagram of a photoelectric sensor in accordance with an embodiment of the present disclosure.

The photoelectric sensor comprises of an emitter and a receiver. In an embodiment, the emitter is a light source. The photoelectric sensors are placed along the borders of the chest wall (202).

In an embodiment, a patient is positioned either in standing or reclining position and the target breast (204) is placed on the chest wall (202) for examination. A person skilled in the art would understand the patient can be placed in any other position which properly places the breast on the chest wall (202). The emitters in the photoelectric sensors emit light and hit the skin line of the breast, that is, the edge of the breast. Once the light hits the breast edge, it is reflected back at the receivers of the sensors at different angles. In an embodiment, multiple photoelectric sensors are used for accurate estimation.

The photoelectric sensor communicates the incident angle to the processor of the mammographic system. The system then compares the incident angle data for the patient under examination with incident angle data stored in a configuration file.

If any portion of the breast (204) is outside of the field of view, the incident angle read by the photoelectric sensor will not match with the incident angle data stored in the configuration file for the corresponding field of view of a patient. The processor (106) then sends a trigger signal to light a red LED on the indicator (110) to alert the operator and prevent breast spread out of the range. The incident angles stored in the configuration file are based on the predefined field of view chosen by the operator based on the size on the breast.

If all the portions of the breast are perfectly within the field of view, the incident angle read by the photoelectric sensor will match with the incident angle data stored in the configuration file for the corresponding field of view of a patient. The system communicates this match event to the processor. The processor then sends a trigger signal to light a green LED indicator to alert the operator that the breast is in the range and mammograms can now be taken. LEDs are placed anywhere on gantry easily visible to the operator from the X-ray console.

In an embodiment, the present disclosure discloses sensing the pressure applied by a patient on the chest wall.

In an embodiment, the present disclosure discloses sensing the positioning of a patient's breast on the chest wall.

In an embodiment, the present disclosure discloses providing an alarm indication when the pressure is beyond a predefined range and/or the positioning of the breast is not proper.

This written description uses examples to disclose the invention, including the best mode, and also to enable any person skilled in the art to practice the invention, including making and using any devices or systems and performing any incorporated methods. The patentable scope of the invention is defined by the claims, and may include other examples that occur to those skilled in the art. Such other examples are intended to be within the scope of the claims if they have structural elements that do not differ from the literal language of the claims, or if they include equivalent structural elements with insubstantial differences from the literal languages of the claims.

What is claimed is:

1. A method of generating an alert while imaging a breast of a subject, the method comprising:
applying a predefined pressure on a breast holder;
confining the breast in a predefined field of view; and
generating an alert based on at least one predefined condition.

2. The method as claimed in claim 1, wherein applying a predefined pressure on a breast holder comprises:
identifying a current pressure applied on the breast holder while imaging; and
comparing the identified pressure with the predefined pressure.

3. The method as claimed in claim 1, wherein the predefined pressure is computed using physical dimensions of the subject with at least one of an operator and a processor.

4. The method as claimed in claim 3, wherein the physical dimensions of the subject are selected from at least one of data stored in a memory unit and data entered by the operator.

5. The method as claimed in claim 2, wherein the breast holder comprises a sensor configured to identify the current pressure applied on the breast holder while imaging.

6. The method as claimed in claim 1, wherein the predefined pressure is different for different subjects.

7. The method as claimed in claim 1, wherein confining the breast in the predefined field of view comprises:
identifying a current field of view of the breast in a compressed position; and
comparing the current field of view with the predefined field of view.

8. The method as claimed in claim 7, wherein the predefined field of view is different for different subjects.

9. The method as claimed in claim 7, wherein identifying a current field of view of the breast in a compressed position comprises:
emitting incident light on the breast with at least one light source;
sensing the light reflected from the breast due to the incident light; and
calculating the angle between the incident light and the reflected light to determine the current field of view.

10. The method as claimed in claim 9, wherein comparing the current field of view and the predefined field of view comprises comparing the calculated angle with an angle defining the predefined field of view.

11. A mammographic system comprising:
at least one sensor configured to determine a current pressure applied on a breast holder while imaging;
a processor configured to compare the current pressure and a predefined pressure; and
an indicator configured to alert an operator of the system based on the comparison of the current pressure and the predefined pressure.

12. The system as claimed in claim 11, wherein the mammographic system is operatively connected to a memory unit, the memory unit being configured to store the predefined pressure and physical dimensions of a subject.

13. The system as claimed in claim 11, wherein the indicator is selected from at least one of a visual indicator and an audio indicator.

14. A mammographic system comprising:
at least one sensor configured to identify a current field of view of a compressed breast while imaging;
a processor configured to compare the current field of view and a predefined field of view; and
an indicator configured to alert an operator of the system based on the comparison of the current field of view and the predefined field of view.

15. The system as claimed in claim 14, wherein the mammographic system is operatively connected to a memory unit, the memory unit being configured to store the predefined field of view and physical dimensions of a subject.

16. The system as claimed in claim 14, wherein the indicator is selected from at least one of a visual indicator and an audio indicator.

17. The system of claim 14, wherein the at least one sensor is a photoelectric sensor.
